(19) United States
(12) Patent Application Publication
(10) Pub. No.: US 2022/0096781 A1
(43) Pub. Date: Mar. 31, 2022

(54) SYNCHRONOUS CONTROL SYSTEMS AND METHODS FOR IMPROVED OXYGEN CONCENTRATION ACCURACY IN BLOWER-BASED VENTILATORS

(71) Applicant: Covidien LP, Mansfield, MA (US)

(72) Inventors: Nancy F. DONG, San Marcos, CA (US); Kun LI, San Diego, CA (US); Gabriel SANCHEZ, Valley Center, CA (US)

(73) Assignee: Covidien LP, Mansfield, MA (US)

(21) Appl. No.: 17/369,382

(22) Filed: Jul. 7, 2021

Related U.S. Application Data
(60) Provisional application No. 63/084,872, filed on Sep. 29, 2020.

(51) Int. Cl. A61M 16/10; A61M 16/00; A61M 16/20; A61M 16/12; G16H 20/40; G16H 40/40; G16H 40/63; G05B 13/02

(52) U.S. Cl. CPC A61M 16/1005; A61M 16/0069; A61M 16/203; A61M 16/125; A61M 2205/3334; G16H 40/40; G16H 40/63; G05B 13/024; A61M 2016/1025; G16H 20/40

(57) ABSTRACT

Systems and methods for increasing accuracy of the fraction of inspired oxygen (FiO2) in delivered breathing gases. In an aspect, the technology relates to a blower-based ventilation system. The system includes a blower; an oxygen flow valve; a processor; and memory storing instructions that, when executed by the processor causes the system to perform a set of operations. The set of operations include, based on a target oxygen concentration level, determining a target ambient air flow rate and a target oxygen flow rate; measuring a flow rate of ambient air generated by a blower; measuring a flow rate of oxygen from an oxygen flow valve; determining a synchronization error; and based on the synchronization error, adjusting operation of at least one of the blower or the oxygen flow valve.

### CROSS-REFERENCE TO RELATED APPLICATIONS

This application claims the benefit of U.S. Provisional Application No. 63/084,872, filed Sep. 29, 2020, the complete disclosure of which is hereby incorporated herein by reference in its entirety.

### INTRODUCTION

Medical ventilator systems are used to provide ventilatory support to patients. Some ventilators include blowers that generate pressurized air to provide to the patients. Depending on the particular condition of a patient, ambient air is enriched with oxygen and the mixture of air is provided to the patient. The oxygen concentration that is desired to be delivered to the patient may depend on the particular patient or condition of the patient.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

Examples of the present disclosure describe systems and methods for increasing accuracy of the fraction of inspired oxygen (FiO2) in delivered breathing gases. In an aspect, the technology relates to a blower-based ventilation system. The system includes a blower; an oxygen flow valve; a processor; and memory storing instructions that, when executed by the processor causes the system to perform a set of operations. The set of operations include: based on a target oxygen concentration level, determining a target ambient air flow rate and a target oxygen flow rate; measuring a flow rate of ambient air generated by a blower; measuring a flow rate of oxygen from an oxygen flow valve; based on the flow rate of ambient air, the flow rate of oxygen, and a ratio of the target ambient air flow rate and the target oxygen flow rate, determining a synchronization error; and based on the synchronization error, adjusting operation of at least one of the blower or the oxygen flow valve.

In an example, the target oxygen concentration level is received as user input into the blower-based ventilation system. In another example, the synchronization error is equal to the flow rate of oxygen subtracted from the product of the ratio and the flow rate of the ambient air. In a further example, determining the target ambient air flow rate and the target oxygen flow rate is further based on a target flow rate for delivered breathing gases. In still another example, the operations further include combining the ambient air generated by the blower and the oxygen from the oxygen flow valve to generate breathing gases; and delivering the breathing gases to a patient, wherein the breathing gases are delivered to the patient at the target flow rate.

In another example, the operations further include based on the synchronization error, generating an adjusted flow value; modifying the target ambient air flow rate based on the adjusted flow value to generate an adjusted target ambient air flow rate; and wherein the adjusting operation of at least one of the blower or the oxygen flow valve includes adjusting the operation of the blower based on the adjusted target ambient flow rate. In still another example, the operations further include based on the synchronization error, generating an adjusted flow value; modifying the target oxygen flow rate based on the adjusted flow value to generate an adjusted target oxygen flow rate; and determining a difference between the flow rate of oxygen and the adjusted target oxygen flow rate; wherein the adjusting operation of at least one of the blower or the oxygen flow valve includes adjusting the operation of the oxygen flow valve based on the adjusted target oxygen flow rate. In still yet another example, the oxygen flow valve is a proportional solenoid (PSOL) valve.

In another aspect, the technology relates to a method for synchronously controlling blower-based ventilation system. The method includes based on a target oxygen concentration level, determining a target ambient air flow rate and a target oxygen flow rate; measuring a flow rate of ambient air generated by a blower; measuring a flow rate of oxygen from an oxygen flow valve; based on the flow rate of ambient air, the flow rate of oxygen, and a ratio of the target ambient air flow rate and the target oxygen flow rate, determining a synchronization error; and based on the synchronization error, adjusting operation of at least one of the blower or the oxygen flow valve.

In an example, the target oxygen concentration level is received as user input into the blower-based ventilation system. In a further example, the synchronization error is equal to the flow rate of oxygen subtracted from the product of the ratio and the flow rate of the ambient air. In another example, determining the target ambient air flow rate and the target oxygen flow rate is further based on a target flow rate for delivered breathing gases. In still another example, combining the ambient air generated by the blower and the oxygen from the oxygen flow valve to generate breathing gases; and delivering the breathing gases to a patient, wherein the breathing gases are delivered to the patient at the target flow rate. In yet another example, an adjusted flow value is generated based on the synchronization error, and at least one of the target ambient air flow rate or the target oxygen flow rate is adjusted by the adjusted flow value. In still yet another example, adjusting the operation of at least one of the blower or the oxygen flow valve is further based on the at least one of the target ambient air flow rate or the target oxygen flow rate adjusted by the adjusted flow value.

In another aspect, the technology relates to a method for synchronously controlling a blower and an oxygen flow valve of a blower-based ventilation system to improve oxygen concentration of delivered breathing gases. The method includes receiving a setting for a target oxygen concentration level (FiO2_{ref}) for delivered breathing gases; accessing a setting for a target flow rate for the delivered breathing gases; based on the target oxygen concentration level (FiO2_{ref}) and the target flow rate, determining a target ambient air flow rate (Q_{air_ref}) and a target oxygen flow (Q_{O2_ref}); operating the blower at a first speed to generate a first flow of ambient air; and opening the oxygen flow valve to a first position to generate a first flow of oxygen through the oxygen flow valve. The method further includes measuring a first air flow rate (Qₐᵢᵣ) of the first flow of ambient air; measuring a first oxygen flow rate (Q_{O2}) of the first flow of oxygen; combining the first flow of ambient air and the first flow of oxygen to generate a first flow of breathing gases; delivering the first flow of breathing gases to a patient, wherein the first flow of breathing gases is delivered at the target flow rate; and based on the measured first air flow rate of ambient air (Qₐᵢᵣ), the measured first oxygen flow rate of oxygen (Q_{O2}), and a ratio of the target ambient air flow rate (Q_{air_ref}) and the target oxygen flow rate (Q_{O2_ref}), determining a synchronization error (ε). The method also includes, based on the synchronization error (ε), operating the blower at a second speed to generate a second flow of ambient air and opening the oxygen flow valve to a second position to generate a second flow of oxygen through the oxygen flow valve; combining the second flow of ambient air and the second flow of oxygen to generate a second flow of breathing gases; and delivering the second flow of breathing gases to a patient, wherein the second flow of breathing gases is delivered at the target flow rate.

In an example, the synchronization error (ε) is equal to Q_{O2_ref}/Q_{air_ref}·Qₐᵢᵣ−Q_{O2}. In another example, the method further includes determining an adjusted flow value (Q_{adjust}), wherein the adjusted flow value (Q_{adjust}) is equal to Q_{adjust}(k)=Kₚ·ε(k)+Kᵢ·Σⱼ₌₁^{k}ε(j), wherein Kₚ and Kᵢ are constant coefficients, the variable j is a control cycle, and the variable k is a current control cycle. In a further example, the setting for the target oxygen concentration level (FiO2_{ref}) is received via an interface of the blower-based ventilation system. In yet another example, the average oxygen concentration error of the first flow of breathing gases and the second flow of breathing gases is less than 5%.

In another aspect, the technology relates to a method for synchronously controlling a blower and an oxygen flow valve of a blower-based ventilation system to improve oxygen concentration of delivered breathing gases. The method includes based on a target oxygen concentration level, determining a target ambient air flow rate and a target oxygen flow rate; measuring a air flow rate of ambient air generated by a blower; measuring a flow rate of oxygen from an oxygen flow valve; based on the flow rate of ambient air, the flow rate of oxygen, and a ratio of the target ambient air flow rate and the target oxygen flow rate, determining a synchronization error; and based on the synchronization error, generating an adjusted flow value. The method also includes modifying the target ambient air flow rate based on the adjusted flow value to generate an adjusted target ambient air flow rate; determining a difference between the flow rate of ambient air and the adjusted target ambient air flow rate; based on the difference between the flow rate of ambient air and the adjusted target ambient air flow rate, adjusting operation of the blower; modifying the target oxygen flow rate based on the adjusted flow value to generate an adjusted target oxygen flow rate; determining a difference between the flow rate of oxygen and the adjusted target oxygen flow rate; and based on the difference between the flow rate of oxygen and the adjusted target oxygen flow rate, adjusting operation of the oxygen flow valve.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

Non-limiting and non-exhaustive examples are described with reference to the following figures.

FIG. 1 depicts an example blower-based ventilation system.

FIG. 2 depicts example additional components of a blower-based ventilation system.

FIG. 3A depicts an example control system for controlling a blower-based ventilation system.

FIG. 3B depicts example plots of measured parameters of ventilation controlled based on the example control system depicted in FIG. 3A.

FIG. 4A depicts an example synchronous control system for synchronously controlling a blower-based ventilation system.

FIG. 4B depicts example plots of measured parameters of ventilation controlled based on the example synchronous control system depicted in FIG. 4A.

FIG. 4C depicts an enlarged portion of the plot of FIG. 4B.

FIGS. 5A-B depict an example method for synchronously controlling a blower-based ventilator.

FIG. 6 depicts another example method for synchronously controlling a blower-based ventilator.

FIGS. 7A-B depict another example method for synchronously controlling a blower-based ventilator.

### DETAILED DESCRIPTION

As discussed above, some ventilators include a blower that provides ambient air into a ventilator and ultimately to a patient. Some of these ventilators may also include an input to receive concentrated oxygen. The concentrated oxygen mixes with the air from the blower to deliver breathing gases to a patient that have a higher concentration of oxygen than the ambient air. The concentration of oxygen in the delivered breathing gases is often referred to as the fraction of inspired oxygen (FiO2).

Depending on the condition of the patient, a medical professional may desire a particular FiO2 value for the patient. To achieve that desired FiO2 level, the amount of additional concentrated oxygen to be added to the breathing gas is controlled. For example, ambient air generally has an oxygen concentration of 21%. Thus, to reach FiO2 levels higher than 21%, additional concentrated oxygen needs to be added to the breathing gases. In many cases, FiO2 levels are desired to be at least 30%, and in some cases 60% or even higher.

The amount of additional oxygen (e.g., in the form of concentrated oxygen gas) that needs to be added to the breathing gases to reach a desired FiO2 level is based on the amount of ambient air being delivered by the blower. Determining that amount of additional oxygen and the timing at which the oxygen is delivered into the breathing gases, however, is a difficult problem in part due to the mechanical components involved. In a simple example, a blower is a fan that generates an air flow by spinning the fan blades. Accordingly, increasing or decreasing the speed of the blower takes some amount of time and is not instantaneous. In contrast, oxygen is often delivered by opening a valve controlling flow of the oxygen from a pressurized oxygen source. The time needed to open and close a valve is significantly less than the time required to spin up or spin down a blower. Due to these operational discrepancies, maintaining a desired FiO2 level is difficult.

Among other things, the present technology addresses the above problems by synchronously controlling the blower and the oxygen valve of the ventilation system, which results in a higher FiO2 accuracy of the delivered breathing gases (e.g., the FiO2 levels of the delivered breathing gases remain closer to the desired FiO2 levels). The synchronous control system of the present technology allows for simultaneously adjusting the operation of the blower and the oxygen valve on a continuous basis to maintain a desired FiO2 level and maintain a target flow of delivered breathing gases. The synchronous and simultaneous control of the blower and the oxygen valve may further be based on an synchronization error, which is based on a ratio of the target ambient air flow and the target oxygen flow. By synchronously controlling both the oxygen valve and the blower in such a manner, the inaccuracies caused by the mechanical and timing differences between the blower and oxygen valve are reduced, and the accuracy of the FiO2 levels of the delivered breathing gases is increased.

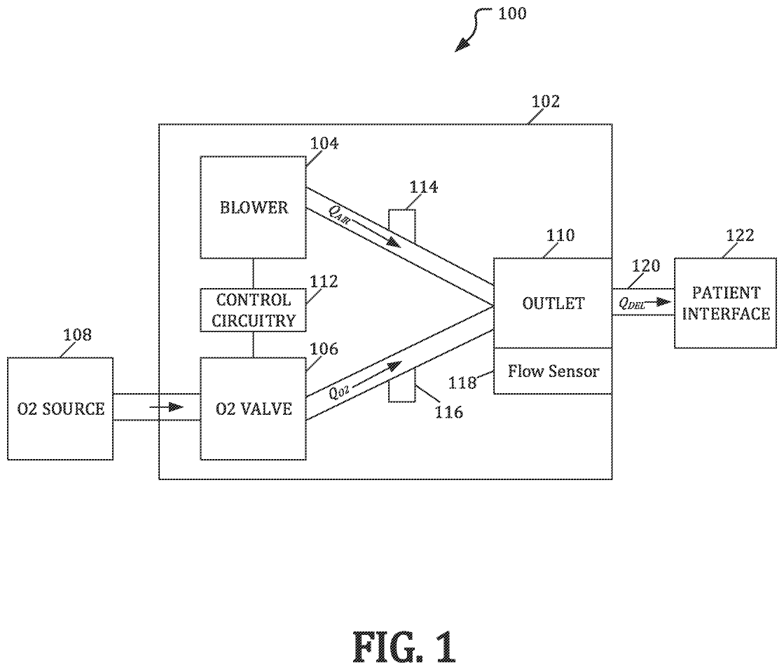

FIG. 1 depicts an example blower-based ventilation system 100. The ventilation system 100 includes a ventilator 102. The ventilator 102 includes a blower 104 and an oxygen flow valve 106. The blower 104 generates air flow of ambient air. The flow rate of a flow of ambient air generated by the blower 104 is represented by the variable Q_{AIR}. The blower 104 includes a fan, and when the fan of the blower 104 spins, ambient air is propelled towards an outlet 110 of the ventilator 102. The flow of the ambient air may be measured by an air flow sensor 114. The air flow sensor 114 may be located at any position sufficient to measure the ambient air flow rate (Q_{AIR}) generated by the blower 104. The oxygen concentration or FiO2 level of the ambient air is approximately the concentration of oxygen of the air outside of the ventilator 102, which is approximately 21%.

The oxygen flow valve 106 controls the flow of concentrated oxygen from an oxygen source 108. The oxygen source 108 may be an oxygen tank that stores pressurized oxygen. In other examples, the oxygen source 108 may include a wall port, such as an oxygen port in a hospital. Depending on the pressure of the oxygen in the oxygen source 108, a regulator may also be incorporated between the oxygen source 108 and the oxygen flow valve 106 to regulate pressure of the oxygen at the oxygen flow valve 106. An opening of the oxygen flow valve 106 allows for oxygen to flow from the oxygen source 108 to the outlet 110 of the ventilator 102. The oxygen valve 106 may be a proportional valve, such as a proportional solenoid (PSOL) valve, that allows for the valve to be set to partially open states in additional to fully open and fully closed states. Accordingly, the amount the oxygen flow valve 106 is open is proportional to the amount of oxygen that flows through the oxygen flow valve 106. The flow rate of the oxygen flowing from the oxygen flow valve 106 is represented by the variable Q_{O2}. The flow rate of oxygen (Q_{O2}) may be measured by an oxygen flow sensor 116. The oxygen flow sensor 116 may be located at any position sufficient to measure the oxygen flow rate (Q_{O2}) released from the oxygen valve 106. The oxygen concentration or FiO2 level of the oxygen may vary between implementations, but for purposes of this disclosure the FiO2 level of the oxygen flowing through the oxygen valve 106 is treated as 100%.

At the outlet 110, the flow of ambient air (Q_{AIR}) and the flow of oxygen (Q_{O2}) are combined and provided through a tubing or breathing circuit 120. The outlet 110 may be an inspiratory port of the ventilator 102. While not depicted, other additional mechanisms that provide for the mixing of the flow of ambient air (Q_{AIR}) and the flow of oxygen (Q_{O2}). The combined flow of ambient air (Q_{AIR}) and the flow of oxygen (Q_{O2}) is referred to delivered flow of breathing gases (Q_{DEL}). The delivered flow of breathing gases (Q_{DEL}) flows through the breathing circuit 120 to a patient interface 122 where the breathing gases are ultimately delivered to the patient. The patient interface 122 may be any suitable interface between the breathing circuit 120 and the patient, such as an endotracheal tube, a mask, a nasal cannula, or similar interfaces.

A flow sensor 118 for the delivered breathing gases may also be included at or near the outlet 110 such that the flow of the delivered breathing gases can be measured. The location of the flow sensor 118 may be any position such that the sensor is within the flow of the combined ambient air and the concentrated oxygen. In some examples, the flow sensor 118 may be located outside the ventilator 102, such as on the breathing circuit 120 and/or at the patient interface 122.

The operation of the blower 104 and the oxygen flow valve 106 may be controlled by control circuitry 112 of the ventilator 102. The control circuitry 112 includes components such as memory and processors that perform the operations described herein. For instance, the control circuitry 112 may include one or more processors and memory. The memory stores instructions that, when executed by the processor(s), causes the ventilator 102, or components thereof, to perform operations described herein. Additional elements of the control circuitry 112 and/or of the ventilation system 100 are described below.

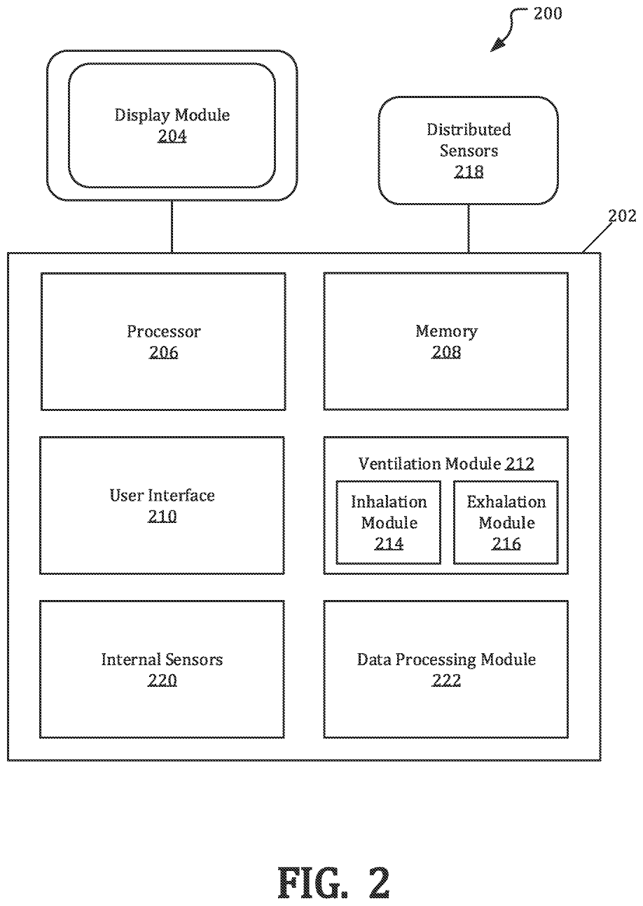

FIG. 2 depicts additional components of a blower-based ventilation system 200. The components of the blower-based ventilation system 200 may be a part of the control circuitry 112 discussed above. Ventilation system 200 includes ventilator 202 with various modules and components. That is, ventilator 202 may further include, among other things, memory 208, one or more processors 206, user interface 210, and ventilation module 212 (which may further include an inhalation module 214 and an exhalation module 216). Processors 206 may be configured with a clock whereby elapsed time may be monitored by the ventilator system 200.

The ventilation system 200 may also include a display module 204 communicatively coupled to ventilator 202. Display module 204 provides various input screens, for receiving input, and various display screens, for presenting useful information. Inputs may also be received from a clinician or other medical professional. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 202 may be provided, for instance by a wheel, keyboard, mouse, or other suitable interactive device. Thus, user interface 210 may accept commands and input through display module 204, such as a baseline flow, PEEP, a high-flow mode, or other parameters related to a particular ventilation mode. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed in the form of graphs, wave representations (e.g., a waveform), pie graphs, numbers, or other suitable forms of graphic display. For example, the data processing module 222 may be operative to control a blower and oxygen valve according to the control algorithms discussed herein.

Ventilation module 212 may control ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with a particular ventilation mode. In some cases, certain ventilation settings may be adjusted based on the exhalation flow, e.g., to adjust or improve the prescribed treatment. Ventilation settings may include inhalation flow, frequency of delivered breaths (e.g., respiratory rate (f), tidal volume (VT), PEEP level, etc.). Ventilation module 212 may further include an inhalation module 214 configured to deliver gases to the patient and an exhalation module 216 configured to receive exhalation gases from the patient, according to ventilation settings that may be based on the exhalation flow. While not depicted in FIG. 1, the ventilator may also include an expiratory port to received breathing gases exhaled from the patient. The operation of the expiratory port may be controlled by the exhalation module 216.

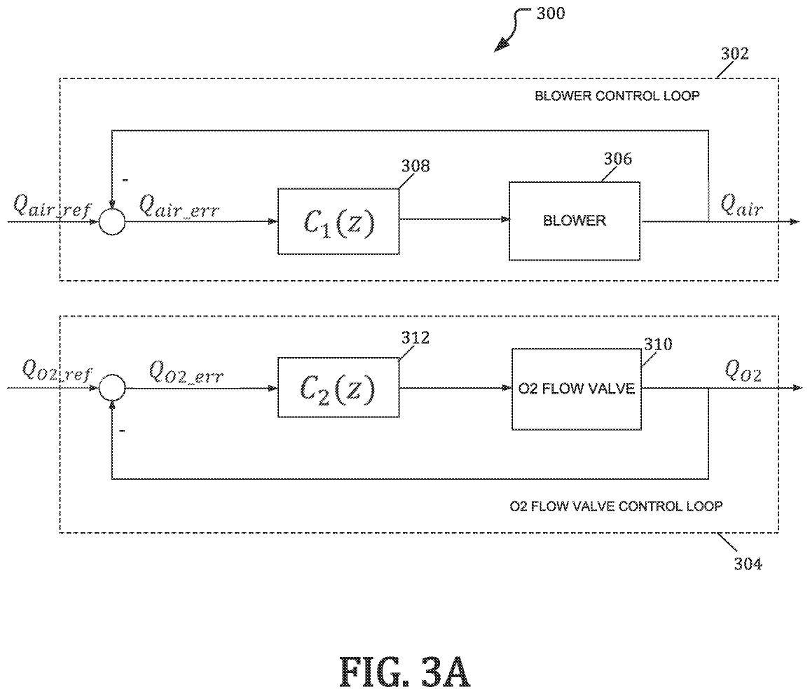

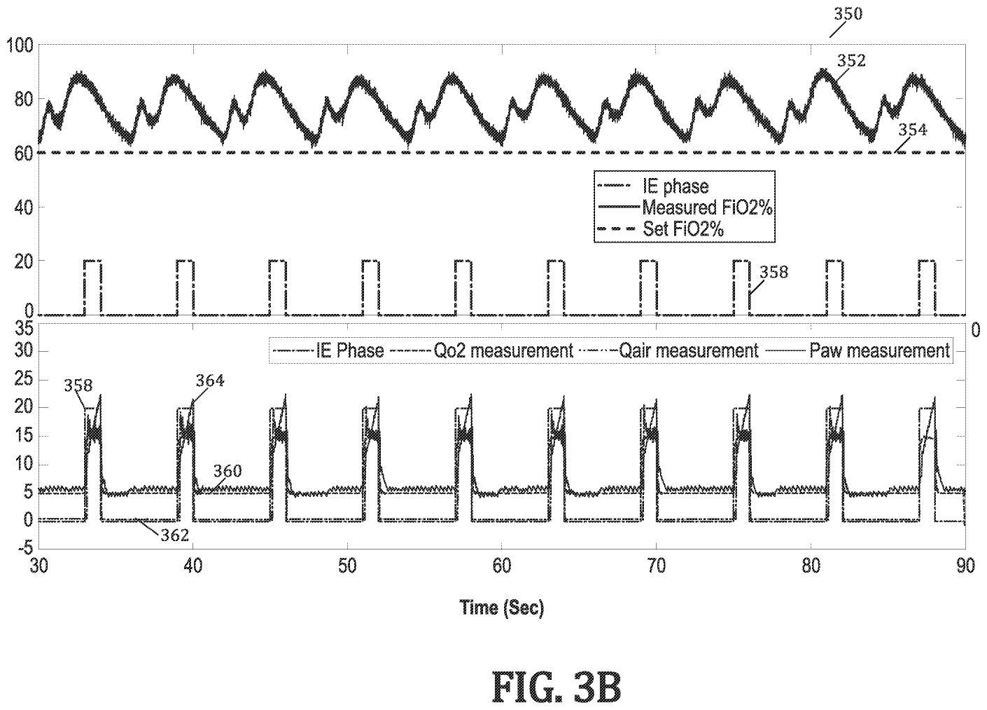

FIG. 3A depicts an example control system 300 for controlling a blower-based ventilation system. The control system 300 includes separate control loops for the blower 306 and the oxygen flow valve 310. More specifically, the control system 300 includes a blower control loop 302 and an oxygen flow valve control loop 304. The blower control loop 302 includes the blower 306 and a discrete time controller (C₁(z)) 308 designed for the blower 306. For the blower control loop 302, the air flow rate of ambient air (Qₐᵢᵣ) from the blower 306 is measured and compared to a target or reference ambient air flow rate (Q_{air_ref}). The difference between the measured air flow rate of ambient air (Qₐᵢᵣ) and the target or reference flow rate of ambient air (Q_{air_ref}) is referred to as the delivered air flow error (Q_{air_err}) and is equal to Q_{air_ref}−Qₐᵢᵣ. Based on the delivered air flow error (Q_{air_err}), the discrete time controller (C₁(z)) 308 may cause an increase or decrease in the speed of the blower 306 to increase or decrease the flow rate of ambient air. For instance, if the delivered air flow error (Q_{air_err}) indicates that the measured flow rate of ambient air (Qₐᵢᵣ) is less than the target ambient air flow rate (Q_{air_ref}), the discrete time controller (C₁(z)) 308 causes an increase in speed of the blower 306 to generate additional flow of ambient air.

The separate oxygen flow valve control loop 304 includes the oxygen flow valve 310 and a discrete time controller (C₂(z)) 312 designed for the oxygen flow valve 310. The flow rate of oxygen (Q_{O2}) through the oxygen flow valve 310 is measured and compared to a target or reference oxygen flow rate (Q_{O2_ref}). The difference between the flow rate of oxygen (Q_{O2}) through the oxygen flow valve 310 and the target or reference oxygen flow rate (Q_{O2_ref}) is referred to as the delivered oxygen flow error (Q_{O2_err}) and is equal to Q_{O2_ref}−Q_{O2}. Based on the delivered oxygen flow error (Q_{O2_err}), the discrete time controller (C₂(z)) 312 may cause the oxygen flow valve 310 to open or close (e.g., move to a more open position or a more closed position) to increase or decrease the flow of oxygen through the oxygen flow valve 310. For example, if the delivered oxygen flow error (Q_{O2_err}) indicates that the measured flow rate of oxygen (Q_{O2}) is less than the target oxygen flow rate (Q_{O2_ref}), the discrete time controller (C₂(z)) 312 causes the oxygen flow valve 310 to open further to allow for additional oxygen to flow through the oxygen flow valve 310.

The target air flow rate (Q_{air_ref}) and the target oxygen flow rate (Q_{O2_ref}) may be determined based on a target FiO2 level (FiO2_{ref}). For the purposes of this disclosure, the oxygen concentration of ambient air is 21% and the oxygen concentration of the oxygen flow is 100%. Of note, in implementations at higher elevations than sea level or difference oxygen concentration values, the preceding numbers should be adjusted accordingly. Thus, for a target flow rate of delivered breathing gases, the proportion of ambient air flow rate to oxygen flow rate can be determined to meet a target or reference FiO2 level. For instance, if the target flow rate of the breathing gases is 30 liters per minute and the target FiO2 level is 47%, then the target air flow rate (Q_{air_ref}) is 20 liters per minute and the target oxygen flow rate (Q_{O2_ref}) is 10 liters per minute. Then, in the control system 300, the blower control loop 302 controls the blower 306 to target the determined target air flow rate (Q_{air_ref}) and, independently, the oxygen flow valve control loop 304 controls the oxygen flow valve 310 to target the determined target oxygen flow rate (Q_{O2_ref}).

In addition, based on the above concepts, the reference FiO2 (FiO2_{ref}) may be expressed as shown below in Equation 1:

$$FiO {2}_{ref} = \frac{0.21 * {Q}_{air \_ ref} + {Q}_{O 2 \_ ref}}{{Q}_{air \_ ref} + {Q}_{O 2 \_ ref}} \cdot 100 \% = 100 \% - \frac{79 \%}{\frac{{Q}_{O 2 \_ ref}}{{Q}_{air \_ ref}} + 1} \qquad ( 1 )$$

Similarly, the measured FiO2 may be expressed as shown below in Equation 2:

$$FiO 2 = \frac{0.21 * {Q}_{air} + {Q}_{O 2}}{{Q}_{air} + {Q}_{O 2}} \cdot 100 \% = 100 \% - \frac{79 \%}{\frac{{Q}_{O 2}}{{Q}_{air}} + 1} \qquad ( 2 )$$

Subtracting Equation 1 from Equation 2 then provides the following equation for the delivered FiO2 (FiO2ₑᵣᵣ) error shown in Equation 3.

$$FiO {2}_{err} = FiO {2}_{ref} - FiO 2 = \frac{79 \%}{\frac{{Q}_{O 2}}{{Q}_{air}} + 1} - \frac{79 \%}{\frac{{Q}_{O 2 \_ ref}}{{Q}_{air \_ ref}} + 1} \qquad ( 3 )$$

Equation 3 implies that if

$$\frac{{Q}_{O 2}}{{Q}_{air}} > \frac{{Q}_{O 2 \_ ref}}{{Q}_{air \_ ref}} ,$$

then FiO2>FiO2_{ref}, and vice versa. That relationship further implies that the oxygen-to-air ratio

$$\frac{{Q}_{O 2}}{{Q}_{air}}$$

affects the accuracy of delivered oxygen concentration FiO2. Because the blower control loop 302 and the oxygen flow valve control loop 304, however, are independent from one another in the control system 300, the two control loops have no knowledge of one another. Therefore, any inaccuracy in one of the control loops is not compensated by the other control loop, which could lead to inaccuracy in FiO2 accuracy. If both the ambient air delivery mechanism and the oxygen delivery mechanism are high-accuracy, quick-responding devices, such as proportional flow valves, the fact that the control loops are independent does not cause significant inaccuracy. However, in examples where the ambient air delivery is controlled by a blower and the oxygen flow is controlled by a valve, the significant operational differences between the two mechanisms leads to FiO2 errors for control loops that are independent from one another, as shown in FIG. 3B and discussed below.

FIG. 3B depicts example plots 305 of measured parameters of ventilation controlled based on the example control system depicted in FIG. 3A. In the upper portion of the plots 350, the measured FiO2 level 352 of the delivered breathing gases and the target, desired, or set FiO2 level are plotted against time. The IE phase signal 358 is also plotted against time. The IE phase signal 358 represents whether the ventilator is in an inspiratory phase or an expiratory phase. When the IE phase signal 358 is high, the ventilator is in an inspiratory phase (e.g., delivering a breath). When the IE phase signal 358 is low, the ventilator is in an expiratory phase.

As can be seen from the plots 350, there is a substantial difference between the measured FiO2 levels 352 of the delivered breathing gases and the set FiO2 level 354. For instance, at some time points the measured FiO2 levels 352 are at roughly 90% where the target FiO2 level 354 is 60%, which results in roughly a 50% deviation from the target FiO2 level 354. The FiO2 error or oxygen concentration error may be calculated by taking the absolute value of the difference between the measured FiO2 level 352 at a time point and the target FiO2 level 354. Accordingly, in the plots 350, the maximum FiO2 error is approximately 30%, the minimum error is approximately 5%, and the average FiO2 error is approximately 15%. In addition, the measured FiO2 level 352 remains above the target FiO2 level 354 at all time points. The reason for both the large difference and the perpetually high FiO2 level is that the oxygen flow is controlled by the high-accuracy oxygen valve and the ambient air flow is controlled by the blower, with each control loop operating independently. For instance, the oxygen valve operates quickly, and the flow of oxygen therefore remains at or close to the target oxygen air flow level. The blower, however, lags behind, which causes the delivered breathing gases to have a higher oxygen concentration.

The bottom portion of the plots 350 plot the IE phase signal 358, the measured oxygen flow rate (Q_{O2}) 360, the measured ambient air flow rate (Qₐᵢᵣ) 362, and the airway pressure (P_{aw}) 364 against time. As can be seen from the plots 350, during an inhalation phase, the measured ambient air flow is a substantially less stable signal than the measured oxygen flow. Such a difference is due to the two different mechanisms controlling the oxygen flow and the ambient air flow.

Due to the inaccuracies that results from the independent control loops in the control system 350, the present technology also includes another control system that is a synchronous control system. The synchronous control system alleviates the substantial difference between the measured FiO2 and the target FiO2 seen in the plots of FIG. 3B. The synchronous control system utilizes the discovery that the oxygen-to-air ratio

$$\frac{{Q}_{O 2}}{{Q}_{air}}$$

affects the accuracy of delivered oxygen concentration FiO2, as discussed above with reference to Equations 1-3. By utilizing a synchronous control system based on the oxygen-to-air ratio, the accuracy of the FiO2 of the delivered breathing gases is improved.

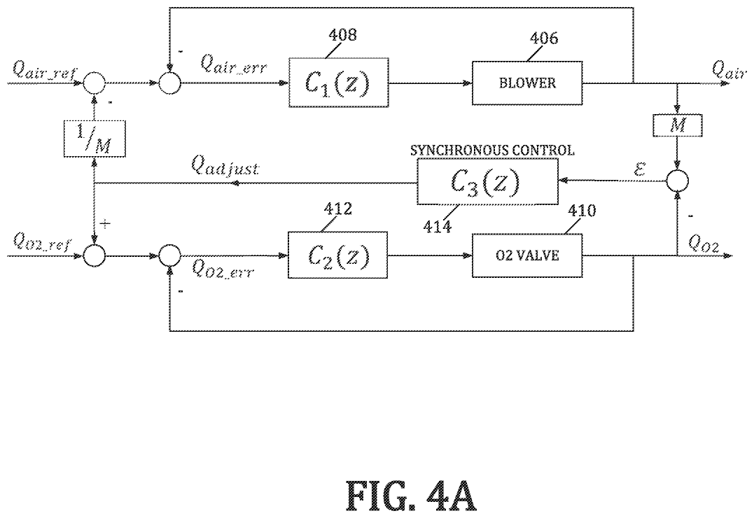

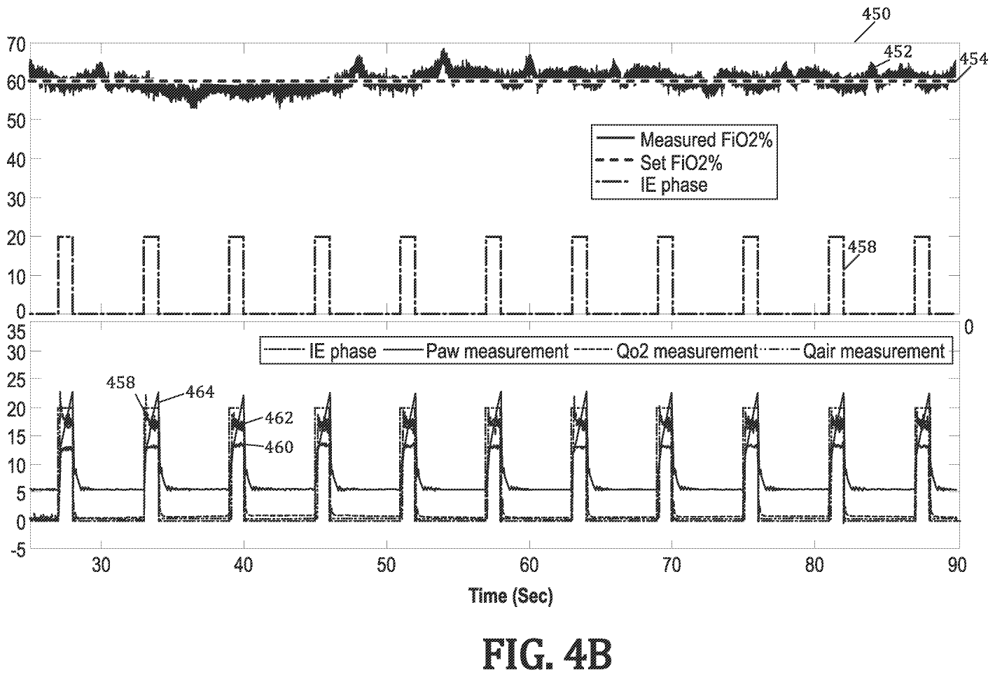

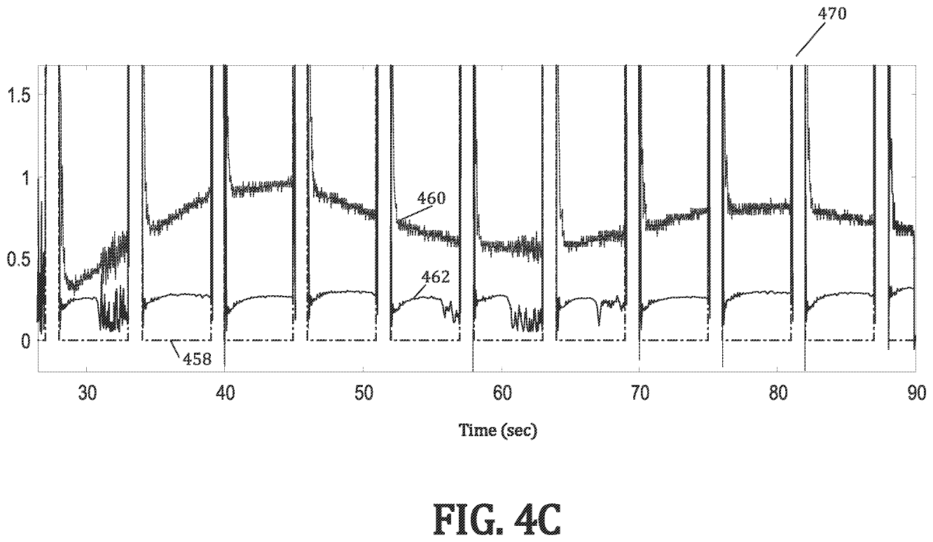

FIG. 4A depicts an example synchronous control system 400 for synchronously controlling a blower-based ventilation system. Similar to the control system 300 depicted in FIG. 3A, the synchronous control system 400 includes a blower 406 and a discrete time controller (C₁(z)) 408 designed for the blower 406. The synchronous control system 400 also includes an oxygen flow valve 410 and a discrete time controller (C₂(z)) 412 designed for controlling the position (e.g., fully open, fully closed, or proportionally open) of the oxygen flow valve 410. Unlike the control system 300 depicted in FIG. 3A, in the synchronous control system 400 none of the control loops are independent from one another, and the synchronous control system 400 includes a third controller (C₃(z)), which may be designed in a discrete time format.

Additional variables are also utilized in the synchronous control system 400. For instance, the desired or target oxygen-to-air ratio

$$\frac{{Q}_{O 2 \_ ref}}{{Q}_{air \_ ref}}$$

is represented by the variable M (i.e.,

$$M = \frac{{Q}_{O 2 \_ ref}}{{Q}_{air \_ ref}} ) .$$

In addition, a synchronization error ε is introduced. The synchronization error ε is equal to the oxygen-to-air ratio (M) multiplied by the difference between the measured ambient air flow (Qₐᵢᵣ) and the measured oxygen flow (Q_{O2}). Thus, the synchronization error ε may be represented by the following Equation 4:

$$ɛ = M \cdot {Q}_{air} - {Q}_{O 2} = \frac{{Q}_{O 2 \_ ref}}{{Q}_{air \_ ref}} \cdot {Q}_{air} - {Q}_{O 2} \qquad ( 4 )$$

Of note, the synchronization error ε indicates the sign of the difference between the measured FiO2 and the target FiO2 (e.g., FiO2−FiO2_{ref}). A synchronization error ε>0 means that FiO2<FiO2_{ref} (i.e., the delivered FiO2 level is lower than the target FiO2 level), and vice versa. The synchronous controller (C₃(z)) 414 then utilizes the synchronization error ε information to adjust the control inputs for both the blower 406 and oxygen flow valve 410.

The synchronous controller (C₃(z)) 414 may be designed as a proportional-integral (PI) controller or a proportional-integral-derivative (PID) controller by utilizing the synchronization error ε. For instance, based on the synchronization error ε, the synchronous controller (C₃(z)) 414 may generate an adjusted flow value Q_{adjust}. As one example, the following Equation 5 may be used as a PI control equation:

Q_{adjust}(k)=Kₚ·ε(k)+Kᵢ·Σⱼ₌₁^{k}ε(j) (5)

In Equation 5, Kₚ and Kᵢ are constant coefficients that may be configured based on the particular type of ventilation system that is implemented. The constant coefficients may be determined experimentally or empirically. For instance, some values for the constant coefficients may cause the synchronous control system 400 to be overly responsive whereas other constants may cause the synchronous control system 400 to be unresponsive and delayed. Regular testing and tuning may be used to select the constant coefficients. The variable j is a control cycle and the variable k is the current control cycle. A control cycle is a cycle at which measurements are taken by sensors and an adjustment value is calculated. In some ventilation systems, the control cycle may be 5 ms, 10 ms, or 20 ms. Other time intervals for control cycles are also possible.

Once the adjusted flow (Q_{adjust}) value is calculated or determined, the Q_{adjust} value is utilized in some examples to adjust both the blower 406 and the oxygen flow valve 410 simultaneously. In other examples, either the blower 406 or the oxygen flow valve 410 may be adjusted based on the determined adjusted flow (Q_{adjust}) value. The determined adjusted flow (Q_{adjust}) value may be used to adjust the target air flow rate (Q_{air_ref}) value prior to the target air flow rate (Q_{air_ref}) value being utilized by the controller (C₁(z)) 408 designed for the blower 406. For instance, the adjusted flow (Q_{adjust}) value may be subtracted from the target air flow rate (Q_{air_ref}) value to generate an adjusted target air flow rate (Q_{air_ref_adjust}) value. The Q_{adjust} value may be multiplied by 1/M prior to being subtracted from the Q_{air_ref} value. A delivered air flow error (Q_{air_err}) between the Q_{air_ref_adjust} value and the measured (Qₐᵢᵣ) value may be determined and utilized by the controller (C₁(z)) 408 to adjust the operation of the blower 406 to target to the Q_{air_ref_adjust} value. For instance, in the synchronous control system 400, the delivered air flow error (Q_{air_err}) is equal to Q_{air_ref_adjust}−Qₐᵢᵣ.

Similarly, the adjusted flow (Q_{adjust}) value may be used to adjust the target oxygen flow rate (Q_{O2_ref}) value prior to the target oxygen flow rate (Q_{O2_ref}) value being utilized by the controller (C₂(z)) 412 designed for the oxygen flow valve 410. For instance, the adjusted flow (Q_{adjust}) value may be added to the target oxygen flow rate (Q_{O2_ref}) value to generate an adjusted target oxygen flow rate (Q_{O2_ref_adjust}) value. A delivered oxygen flow error (Q_{O2_err}) between the adjusted target oxygen flow rate (Q_{O2_ref_adjust}) value and the measured oxygen flow rate (Q_{O2}) value may be determined and utilized by the controller (C₂(z)) 412 to adjust the operation of the oxygen flow valve 410 to target to the Q_{O2_ref_adjust} value. For instance, in the synchronous control system 400, the delivered oxygen flow error (Q_{O2_err}) is equal to Q_{O2_ref_adjust}−Q_{O2}. By synchronously controlling the blower and the oxygen flow valve in such a manner, the accuracy of the FiO2 values of the delivered breathing gases is improved, and the improved FiO2 accuracy can be seen in the plots of FIG. 4B, discussed below.

FIG. 4B depicts example plots 450 of measured parameters of ventilation controlled based on the example synchronous control system depicted in FIG. 4A. In the upper portion of the plots 450, the measured FiO2 level 452 of the delivered breathing gases and the target or set FiO2 level 454 are plotted against time. The IE phase signal 458 is also plotted against time. The IE phase signal 458 represents whether the ventilator is in an inspiratory phase or an expiratory phase. When the IE phase signal 458 is high, the ventilator is in an inspiratory phase (e.g., delivering a breath). When the IE phase signal 458 is low, the ventilator is in an expiratory phase.

As can be seen from the plots 450, the measured FiO2 levels 452 remain quite close to the target FiO2 level 354. For instance, the maximum deviation over the time period plotted is roughly a difference of 8% FiO2 (which occurs at roughly 54 seconds). In terms of FiO2 error, in the plots 450, the maximum FiO2 error is approximately 8%, the minimum error is approximately 0%, and the average FiO2 error is less than approximately 2%. In some examples, synchronous control system 400 is generally able to maintain an average FiO2 error of less than 5%, however, in other examples the average FiO2 error is even less, such as shown in plots 450.

As compared to the plots 350 in FIG. 3B using the non-synchronous control system of FIG. 3A, the FiO2 accuracy in plots 450 using the synchronous control system of FIG. 4A is significantly better. For example, the deviation of measured FiO2 452 from the target FiO2 level 454 is substantially less and the average measured FiO2 level 452 is substantially closer to the target FiO2 level 454. The improvement in accuracy is due to the synchronous control system that allows for correction or adjustment of the oxygen flow valve based on performance of the blower, and vice versa.

The bottom portion of the plots 450 plot the IE phase 458, the measured oxygen flow rate (Q_{O2}) 460, the measured ambient air flow rate (Qₐᵢᵣ) 462, and the airway pressure (P_{aw}) 464 against time. FIG. 4C depicts an enlarged portion 470 of the bottom portion of the plots 450 of FIG. 4B. In FIG. 4C, the synchronization of the blower and the oxygen flow valve can be more clearly seen. For instance, for the exhalation phase between about 58-63 seconds, the measured oxygen flow (Q_{O2}) 460 and the measured ambient air flow (Qₐᵢᵣ) 462 oscillate in response to one another. This behavior is due to the synchronous control system of FIG. 4A being used to synchronously control the blower and the oxygen flow valve. Accordingly, by having the blower and the oxygen flow valve synchronously respond to an error in FiO2, both mechanisms can be adjusted to better maintain the FiO2 levels of the delivered breathing gases at the target FiO2 level.

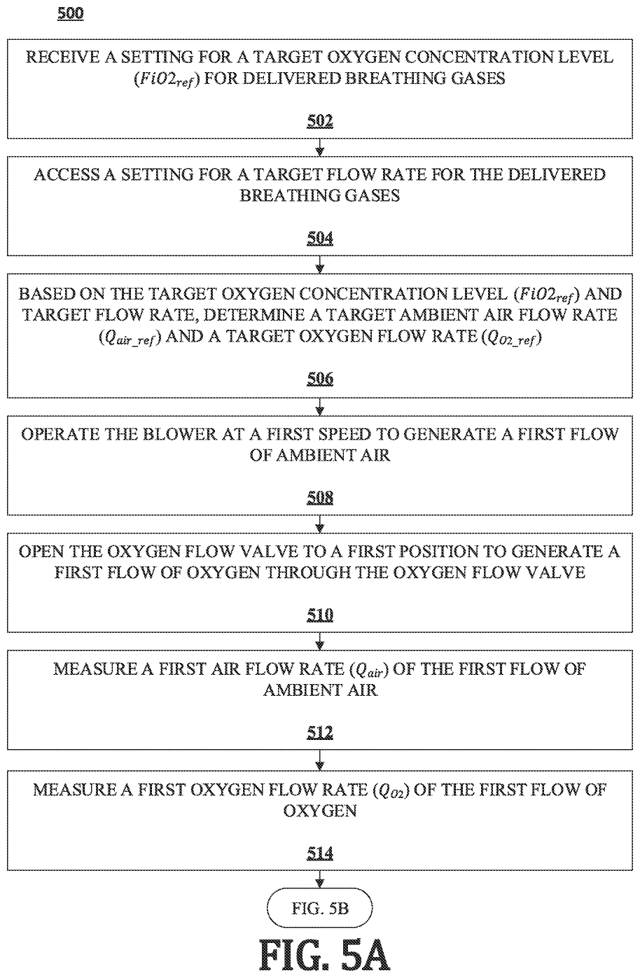

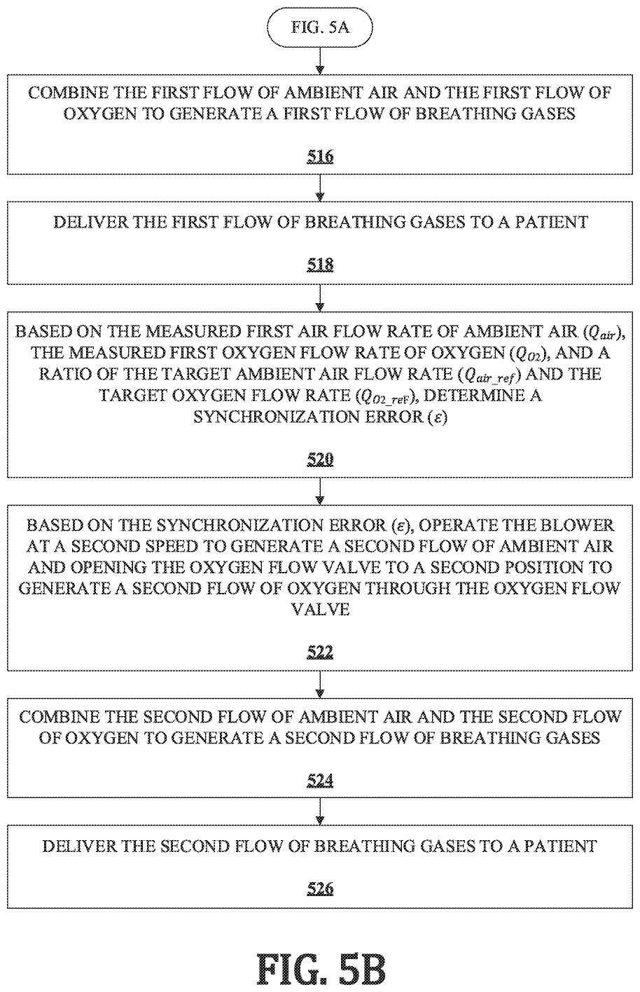

FIGS. 5A-B depicts an example method 500 for synchronously controlling a blower-based ventilator. At operation 502, a setting for a target oxygen concentration level (FiO2_{ref}) is received. The setting may be received via an input at the blower-based ventilator. For instance, the setting may be entered via a touchscreen or another type of input (e.g., button, wheel, dial, keyboard, etc.). The target oxygen concentration level (FiO2_{ref}) may also be received based on calculations or automatic determinations based on the condition of the patient and/or a ventilation mode selected or implemented. At operation 504, a setting for a target flow rate for the delivered breathing gases is accessed and/or received. The target flow rate may change during the course of ventilation. For instance, during an inspiratory phase of a breath, the target flow rate is higher than the target flow rate during an expiratory phase of a breath. The target flow rate may also change during an inspiratory and/or expiratory phase of a breath depending on the ventilation mode. Accordingly, the ventilator system may calculate the target flow rate on a continuous or semi-continuous basis for some ventilation modes and depending on the phase of the breath. The target flow rate may also be received via an input to the ventilator, such as from a medical professional.

At operation 506, a target ambient air flow rate (Q_{air_ref}) and a target oxygen flow rate (Q_{O2_ref}) are determined based on the target oxygen concentration level (FiO2_{ref}) received in operation 502 and the target flow rate accessed in operation 504. For the purpose of this disclosure, as discussed above, the oxygen concentration of ambient air is assumed to be about 21% and the oxygen concentration of the oxygen flow is 100%. Thus, for a target flow of delivered breathing gases, the proportion of ambient air flow to oxygen flow is determined to meet a target oxygen concentration level (FiO2_{ref}) and a target flow rate. For instance, if the target flow rate of the breathing gases is 30 liters per minute and the target FiO2 level is 47%, then the target air flow rate (Q_{air_ref}) is 20 liters per minute and the target oxygen flow rate (Q_{O2_ref}) is 10 liters per minute. Based on the target air flow rate (Q_{air_ref}) and the target oxygen flow rate (Q_{O2_ref}), the control systems are able to control the blower and the oxygen flow valve to target the target flow rates.

At operation 508, the ventilation system operates the blower at a first speed to generate a first flow of ambient air. For instance, the fan of the blower is operated at a first speed, which propels a first flow of ambient air. At operation 510, the ventilation system opens the oxygen flow valve to a first position to generate a first flow of oxygen through the oxygen flow valve. For example, by opening the oxygen flow valve, pressurized oxygen from an oxygen source is allowed to flow through the oxygen flow valve. The rate and quantity of oxygen allowed to flow through the oxygen flow valve may be based on a proportion that the oxygen flow valve is open.

At operation 512, a first air flow rate (Qₐᵢᵣ) of the first flow of ambient air, generated in operation 508, is measured. The first air flow rate (Qₐᵢᵣ) may be measured by a flow sensor or other similar sensor that is positioned in the ventilation system so as to measure the first air flow rate (Qₐᵢᵣ). At operation 514, a first oxygen flow rate (Q_{O2}) of the first flow of oxygen, generated by opening the oxygen flow valve in operation 510, is measured. The first oxygen flow rate (Q_{O2}) may be measured by a flow sensor or other similar sensor that is positioned in the ventilation system so as to measure the first oxygen flow rate (Q_{O2}).

At operation 516, the first flow of ambient air and the first flow of oxygen are combined to generate a first flow of breathing gases. Combining the first flow of ambient air and the first flow of oxygen may be performed passively. For instance, the tubing for the ambient air and the tubing for the oxygen may be connected to a tubing system for the breathing gases that are to be delivered. Such a combination may occur at or near the outlet of the ventilator. At operation 518, the first flow of breathing gases generated in operation 516 is delivered to a patient. Delivery of the breathing gases to the patient may include providing the breathing gases to a patient circuit and a patient interface. The breathing gases may be delivered at the target flow rate accessed in operation 504.

At operation 520, a synchronization error (ε) is determined. The synchronization error (ε) is determined based on the measured first air flow rate of ambient air (Qₐᵢᵣ), the measured first oxygen flow rate of oxygen (Q_{O2}), and a ratio of the target ambient air flow rate (Q_{air_ref}) and the target oxygen flow rate (Q_{O2_ref}). The synchronization error may be equal to the first oxygen flow rate of oxygen (Q_{O2}) subtracted from the product of the ratio and measured first air flow rate of ambient air (Qₐᵢᵣ). The ratio may be the oxygen-to-air ratio M, which is equal to

$$\frac{{Q}_{O 2 \_ ref}}{{Q}_{air \_ ref}} .$$

The synchronization error (ε) may be calculated according to the following Equation 6:

$$ɛ = M \cdot {Q}_{air} - {Q}_{O 2} = \frac{{Q}_{O 2 \_ ref}}{{Q}_{air \_ ref}} \cdot {Q}_{air} - {Q}_{O 2} \qquad ( 6 )$$

At operation 522, the operation of the blower and the operation of the oxygen valve are changed or adjusted based on the synchronization error (ε) determined in operation 520. For instance, based on the synchronization error (ε), the blower may be operated at a second speed to generate a second flow of ambient air having a second air flow rate. In addition, also based on the synchronization error (ε), the oxygen flow valve may be operated such that in opens to a second position to generate a second flow of oxygen through the oxygen flow valve. The second flow of oxygen may have a second oxygen flow rate. The blower and the oxygen flow valve may be adjusted in opposite manners based on the synchronization error (ε). For instance, in some examples, the speed of the blower may be increased to increase the flow rate of ambient air and the oxygen flow valve may be moved to a more closed position to reduce the flow rate of oxygen. Accordingly, the combined flow rate of the second air flow rate and the second oxygen flow rate may be substantially the same as the combined flow rate of the first air flow rate and the second air flow rate. The adjustments to the blower and the oxygen flow valve may be performed utilizing the example synchronous control system of FIG. 4A and/or the concepts described herein.

At operation 524, the second flow of ambient air and the second flow of oxygen are combined to generate a second flow of breathing gases. The second flow of ambient air and the second flow of oxygen may be combined in the same or similar manner as the combination of flows in operation 516. At operation 526, the second flow of breathing gases is delivered to the patient. The second flow of breathing gases may be delivered in the same or similar manner as the first flow of breathing gases delivered in operation 518. The second flow of breathing gases may be delivered at the target flow rate accessed in operation 504.

Method 500 may repeat on any desired interval, such as every control cycle of the ventilator. Accordingly, the operation of the blower and oxygen flow valve may be continuously and synchronously adjusted to improve FiO2 accuracy while maintaining desired target flow rates for the delivered breathing gases.

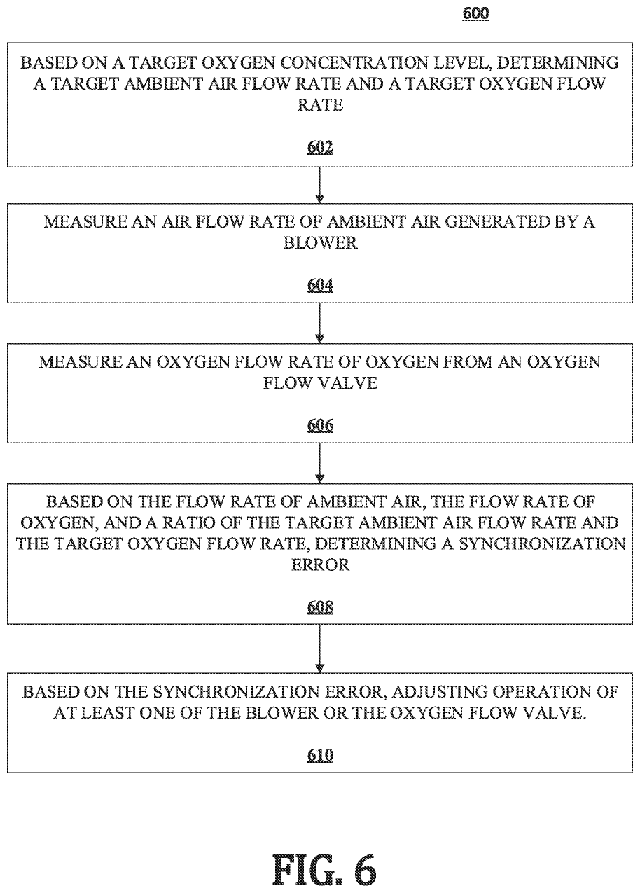

FIG. 6 depicts another example method 600 for synchronously controlling a blower-based ventilator. At operation 602, a target ambient air flow rate and a target oxygen flow rate are determined based on a target oxygen concentration level. The target ambient air flow rate and a target oxygen flow rate may also be determined based on a target flow rate for delivered breathing gases.

At operation 604, an air flow rate of an ambient air flow generated by the blower is measured. The air flow rate may be measured by a flow sensor or other similar sensor that is positioned in the ventilation system so as to measure the oxygen flow rate. At operation 606, an oxygen flow rate of an oxygen flow flowing from the oxygen flow valve is measured. The oxygen flow rate may be measured by a flow sensor or other similar sensor that is positioned in the ventilation system so as to measure the oxygen flow rate.

At operation 608, a synchronization error is determined based on the air flow rate of the ambient air measured in operation 604, the oxygen flow rate measured in operation 606, and a ratio of the target ambient flow rate and the target oxygen flow rate determined in operation 602. The synchronization error may be equal to the oxygen flow rate subtracted from the product of the ratio and the air flow rate of ambient air. The ratio may be the oxygen-to-air ratio M discussed above and the synchronization error may be determined using Equation 4, above.

At operation 610, the operation of the blower and/or the oxygen flow valve may be adjusted based on the synchronization error determined in operation 608. For instance, based on the synchronization error (ε), the blower may be operated at an adjusted speed to generate a different flow of ambient air. In addition or alternatively, also based on the synchronization error (ε), the oxygen flow valve may be operated such that in opens to a different position to generate a different flow of oxygen through the oxygen flow valve. The blower and the oxygen flow valve may be adjusted in opposite manners based on the synchronization error (ε). For instance, in some examples, the speed of the blower may be increased to increase the flow rate of ambient air and the oxygen flow valve may be moved to a more closed position to reduce the flow rate of oxygen. Accordingly, the combined flow rate of the second air flow rate and the second oxygen flow rate may be substantially the same as the combined flow rate of the first air flow rate and the second air flow rate. The adjustments to the blower and the oxygen flow valve may be performed utilizing the example synchronous control system of FIG. 4A and/or the concepts described herein.

Method 600 may repeat on any desired interval, such as every control cycle of the ventilator. Accordingly, the operation of the blower and oxygen flow valve may be continuously and synchronously adjusted to improve FiO2 accuracy while maintaining desired target flow rates for the delivered breathing gases.

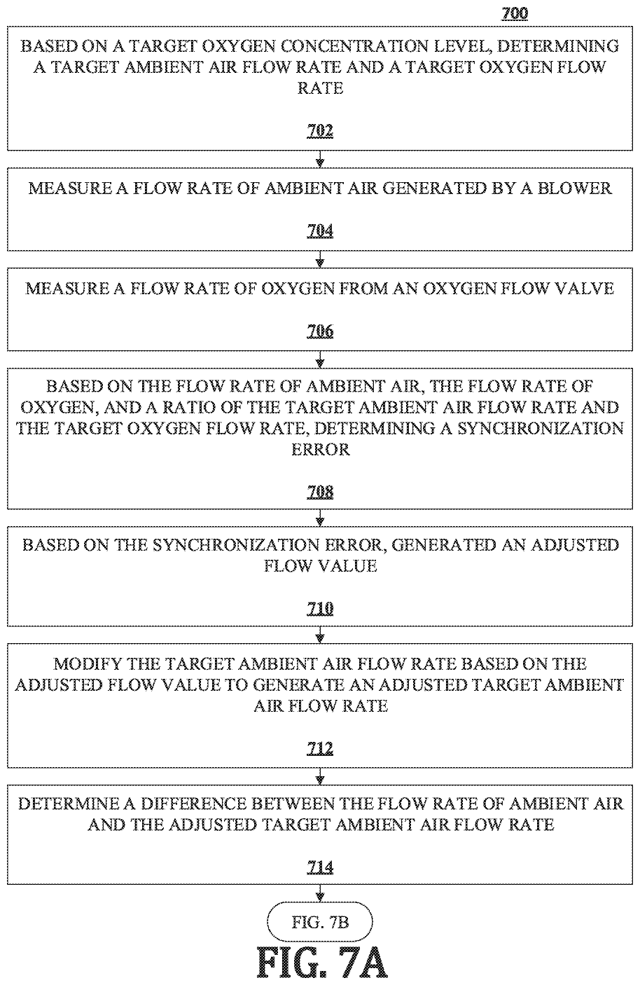

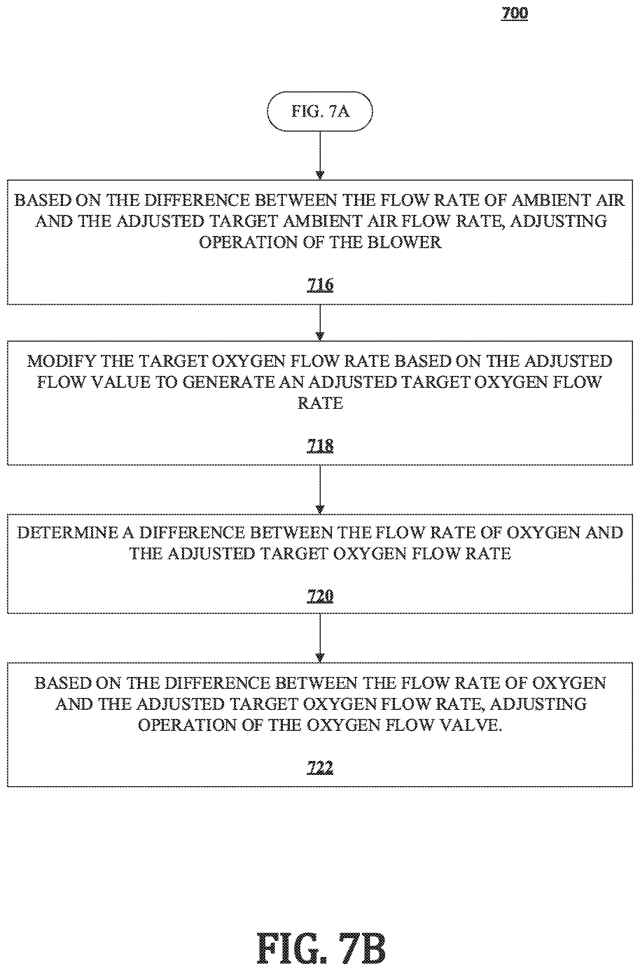

FIGS. 7A-B depict another example method 700 for synchronously controlling a blower-based ventilator. At operation 702, a target ambient air flow rate and a target oxygen flow rate are determined based on a target oxygen concentration level. The target ambient air flow rate and a target oxygen flow rate may also be determined based on a target flow rate for delivered breathing gases.

At operation 704, an air flow rate of an ambient air flow generated by the blower is measured. The air flow rate may be measured by a flow sensor or other similar sensor that is positioned in the ventilation system so as to measure the oxygen flow rate. At operation 706, an oxygen flow rate of an oxygen flow flowing from the oxygen flow valve is measured. The oxygen flow rate may be measured by a flow sensor or other similar sensor that is positioned in the ventilation system so as to measure the oxygen flow rate.

At operation 708, a synchronization error is determined based on the air flow rate of the ambient air measured in operation 704, the oxygen flow rate measured in operation 706, and a ratio of the target ambient flow rate and the target oxygen flow rate determined in operation 702. The synchronization error may be equal to the oxygen flow rate subtracted from the product of the ratio and the air flow rate of ambient air. The ratio may be the oxygen-to-air ratio M discussed above and the synchronization error may be determined using Equation 4, above.

At operation 710, an adjusted flow value is generated based on the synchronization error determined in operation 708. The adjusted flow value may be the Q_{adjust} value discussed above. The adjusted flow value may be determined by a PI or PID controller and may be calculated according to Equation 5, above.

At operation 712, the target ambient air flow rate is modified based on the adjusted flow value to generate an adjusted target ambient air flow rate. The adjusted target ambient air flow rate may be generated by subtracting the adjusted flow value from the target ambient air flow rate. In some examples, prior to the adjusted flow value being subtracted from the target ambient air flow rate, the adjusted flow value is multiplied by the inverse of the ratio (e.g., 1/m).

At operation 714, a difference is determined between the measured flow rate of ambient air measured in operation 704 and the adjusted target ambient air flow rate determined in operation 712. At operation 716, based on the difference determined in operation 714, the operation of the blower is adjusted. For instance, the speed of the blower may be increased to increase the ambient air flow rate, or the speed of the blower may be decreased to decrease the ambient air flow rate.

At operation 718, the target oxygen flow rate is modified based on the adjusted flow value to generate an adjusted target oxygen flow rate. The adjusted oxygen flow rate may be generated by adding the adjusted flow value to the target oxygen flow rate. At operation 720, a difference is determined between the measured flow rate of oxygen measured in operation 706 and the adjusted target oxygen flow rate determined in operation 718. At operation 722, based on the difference determined in operation 720, the operation of the oxygen flow valve is adjusted. For instance, the position of the valve (e.g., the amount the valve is opened) may be adjusted to increase or decrease the oxygen flow rate.

Method 700 may repeat on any desired interval, such as every control cycle of the ventilator. Accordingly, the operation of the blower and oxygen flow valve may be continuously and synchronously adjusted to improve FiO2 accuracy while maintaining desired target flow rates for the delivered breathing gases.

The embodiments described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices may be employed to perform the functionality disclosed herein without departing from the scope of the disclosure. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

This disclosure describes some embodiments of the present technology with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art. Further, as used herein and in the claims, the phrase “at least one of element A, element B, or element C” is intended to convey any of: element A, element B, element C, elements A and B, elements A and C, elements B and C, and elements A, B, and C. Further, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Although specific embodiments are described herein, the scope of the technology is not limited to those specific embodiments. Moreover, while different examples and embodiments may be described separately, such embodiments and examples may be combined with one another in implementing the technology described herein. One skilled in the art will recognize other embodiments or improvements that are within the scope and spirit of the present technology. Therefore, the specific structure, acts, or media are disclosed only as illustrative embodiments. The scope of the technology is defined by the following claims and any equivalents therein.

What is claimed is:

1. A blower-based ventilation system, the system comprising:
a blower;
an oxygen flow valve;
a processor; and
memory storing instructions that, when executed by the processor causes the system to perform a set of operations:
based on a target oxygen concentration level, determining a target ambient air flow rate and a target oxygen flow rate;
measuring a flow rate of ambient air generated by a blower;
measuring a flow rate of oxygen from an oxygen flow valve;
based on the flow rate of ambient air, the flow rate of oxygen, and a ratio of the target ambient air flow rate and the target oxygen flow rate, determining a synchronization error; and
based on the synchronization error, adjusting operation of at least one of the blower or the oxygen flow valve.

2. The system of claim 1, wherein the target oxygen concentration level is received as user input into the blower-based ventilation system.

3. The system of claim 1, wherein the synchronization error is equal to the flow rate of oxygen subtracted from the product of the ratio and the flow rate of the ambient air.

4. The system of claim 1, wherein determining the target ambient air flow rate and the target oxygen flow rate is further based on a target flow rate for delivered breathing gases.

5. The system of claim 4, wherein the operations further comprise:
combining the ambient air generated by the blower and the oxygen from the oxygen flow valve to generate breathing gases; and
delivering the breathing gases to a patient, wherein the breathing gases are delivered to the patient at the target flow rate.

6. The system of claim 1, wherein the operations further comprise:
based on the synchronization error, generating an adjusted flow value;
modifying the target ambient air flow rate based on the adjusted flow value to generate an adjusted target ambient air flow rate; and
wherein the adjusting operation of at least one of the blower or the oxygen flow valve includes adjusting the operation of the blower based on the adjusted target ambient flow rate.

7. The system of claim 1, wherein the operations further comprise:
based on the synchronization error, generating an adjusted flow value;
modifying the target oxygen flow rate based on the adjusted flow value to generate an adjusted target oxygen flow rate; and
determining a difference between the flow rate of oxygen and the adjusted target oxygen flow rate;
wherein the adjusting operation of at least one of the blower or the oxygen flow valve includes adjusting the operation of the oxygen flow valve based on the adjusted target oxygen flow rate.

8. The system of claim 1, wherein the oxygen flow valve is a proportional solenoid (PSOL) valve.

9. A method for synchronously controlling blower-based ventilation system, the method comprising:
based on a target oxygen concentration level, determining a target ambient air flow rate and a target oxygen flow rate;
measuring a flow rate of ambient air generated by a blower;
measuring a flow rate of oxygen from an oxygen flow valve;
based on the flow rate of ambient air, the flow rate of oxygen, and a ratio of the target ambient air flow rate and the target oxygen flow rate, determining a synchronization error; and
based on the synchronization error, adjusting operation of at least one of the blower or the oxygen flow valve.

10. The method of claim 9, wherein the target oxygen concentration level is received as user input into the blower-based ventilation system.

11. The method of claim 9, wherein the synchronization error is equal to the flow rate of oxygen subtracted from the product of the ratio and the flow rate of the ambient air.

12. The method of claim 9, wherein determining the target ambient air flow rate and the target oxygen flow rate is further based on a target flow rate for delivered breathing gases.

13. The method of claim 12, further comprising:
combining the ambient air generated by the blower and the oxygen from the oxygen flow valve to generate breathing gases; and
delivering the breathing gases to a patient, wherein the breathing gases are delivered to the patient at the target flow rate.

14. The method of claim 9, wherein an adjusted flow value is generated based on the synchronization error, and at least one of the target ambient air flow rate or the target oxygen flow rate is adjusted by the adjusted flow value.

15. The method of claim 14, wherein adjusting the operation of at least one of the blower or the oxygen flow valve is further based on the at least one of the target ambient air flow rate or the target oxygen flow rate adjusted by the adjusted flow value.

16. A method for synchronously controlling a blower and an oxygen flow valve of a blower-based ventilation system to improve oxygen concentration of delivered breathing gases, the method comprising:
receiving a setting for a target oxygen concentration level (FiO2_{ref}) for delivered breathing gases;
accessing a setting for a target flow rate for the delivered breathing gases;
based on the target oxygen concentration level (FiO2_{ref}) and the target flow rate, determining a target ambient air flow rate (Q_{air_ref}) and a target oxygen flow (Q_{O2_ref});
operating the blower at a first speed to generate a first flow of ambient air;
opening the oxygen flow valve to a first position to generate a first flow of oxygen through the oxygen flow valve;
measuring a first air flow rate (Qₐᵢᵣ) of the first flow of ambient air;
measuring a first oxygen flow rate (Q_{O2}) of the first flow of oxygen;
combining the first flow of ambient air and the first flow of oxygen to generate a first flow of breathing gases;
delivering the first flow of breathing gases to a patient, wherein the first flow of breathing gases is delivered at the target flow rate;
based on the measured first air flow rate of ambient air (Qₐᵢᵣ), the measured first oxygen flow rate of oxygen (Q_{O2}), and a ratio of the target ambient air flow rate (Q_{air_ref}) and the target oxygen flow rate (Q_{O2_ref}), determining a synchronization error (ε);
based on the synchronization error (ε), operating the blower at a second speed to generate a second flow of ambient air and opening the oxygen flow valve to a second position to generate a second flow of oxygen through the oxygen flow valve;
combining the second flow of ambient air and the second flow of oxygen to generate a second flow of breathing gases; and
delivering the second flow of breathing gases to a patient, wherein the second flow of breathing gases is delivered at the target flow rate.

17. The method of claim 16, wherein the synchronization error (ε) is equal to
$$\frac{{Q}_{O 2 \_ ref}}{{Q}_{air \_ ref}} \cdot {Q}_{air} - {Q}_{O 2} .$$

18. The method of claim 16, further comprising determining an adjusted flow value (Q_{adjust}), wherein the adjusted flow value (Q_{adjust}) is equal to Q_{adjust}(k)=Kₚ·ε(k)+Kᵢ·Σⱼ₌₁^{k}ε(j) wherein Kₚ and Kᵢ are constant coefficients, the variable j is a control cycle, and the variable k is a current control cycle.

19. The method of claim 16, wherein the setting for the target oxygen concentration level (FiO2_{ref}) is received via an interface of the blower-based ventilation system.

20. The method of claim 16, wherein the average oxygen concentration error of the first flow of breathing gases and the second flow of breathing gases is less than 5%.
